(19) United States
(12) Patent Application Publication
(10) Pub. No.: US 2024/0398220 A1
(43) Pub. Date: Dec. 5, 2024

(54) ENDOSCOPIC SYSTEMS AND METHODS FOR TREATING HEARING LOSS

(71) Applicant: Spiral Therapeutics Inc., South San Francisco, CA (US)

(72) Inventors: Signe Erickson, South San Francisco, CA (US); Vrad Levering, Brisbane, CA (US); Eugene de Juan, South San Francisco, CA (US)

(21) Appl. No.: 18/697,137

(22) PCT Filed: Sep. 12, 2022
(86) PCT No.: PCT/US2022/043187

§ 371 Date: Mar. 29, 2024

Related U.S. Application Data
(60) Provisional application No. 63/249,803, filed on Sep. 29, 2021.

(51) Int. Cl. A61B 1/227; A61B 1/018; A61M 25/00

(52) U.S. Cl. CPC A61B 1/227; A61B 1/018; A61M 2025/009; A61M 25/0097; A61M 2210/0668

(57) ABSTRACT

Systems and methods can be employed for trans-tympanic membrane access to the middle ear for delivery of a therapeutic agent, for example, to the round window niche adjacent to the cochlea under direct visualization. The systems and methods can also be used to improve accessibility and visualization for various other otological surgical procedures.

### TECHNICAL FIELD

This document relates to systems, methods, and materials for treating ear disorders including, but not limited to, hearing loss. In some examples, the systems and methods include trans-tympanic membrane access to the middle ear for targeted delivery of a therapeutic formulation under direct visualization.

### BACKGROUND

The human ear is subject to a variety of disorders including, but not limited to, hearing loss, tinnitus, balance disorders including vertigo, Meniere's Disease, vestibular neuronitis, vestibular schwannoma, labyrinthitis, otosclerosis, ossicular chain dislocation, cholesteatoma, outer ear infections, middle ear infections, schwannoma, and tympanic membrane perforations, to provide a few examples.

In one example, Conductive Hearing Loss (CHL) involves the loss of normal mechanical pathways for sound to reach the hair cells in the cochlea, for example due to malformation, accumulation of fluid in the middle ear, disruption of the tympanic membrane, presence of tumors, and/or damage to ossicles. SensoriNeural Hearing Loss (SNHL) is due to the absence of, or damage to, hair cells or other structures in the cochlea, or to the acoustic nerve. SNHL is typically associated with exposure to loud noise, head trauma, aging, infection, Meniere's Disease, tumors, ototoxicity, genetic diseases like Usher's disease, and the like.

### SUMMARY

This document describes systems and methods for minimally invasive access to the middle ear for the purposes of delivering treatment for inner and middle ear disorders. For example, this document describes systems and methods for trans-tympanic membrane instrument access to achieve minimally invasive delivery of therapeutic formulations into a round window niche adjacent to a round window membrane of a cochlea under direct visualization. In particular implementations, the active agent of the therapeutic formulation may then transfer passively by diffusion across the round window membrane(s), according to a concentration gradient, into the perilymph (within the cochlea).

The devices, systems, materials, compounds, compositions, articles, and methods described herein may be used to treat a variety of disorders of the middle ear and/or inner ear including, but not limited to, hearing loss, tinnitus, balance disorders including vertigo, Meniere's Disease, vestibular neuronitis, vestibular schwannoma, labyrinthitis, otosclerosis, ossicular chain dislocation, cholesteatoma, middle ear infections, and tympanic membrane perforations, to provide a few examples. The devices, systems, materials, compounds, compositions, articles, and methods described herein may also be used to locally deliver a nerve numbing agent (e.g., Botox®) to treat stapedius tremors. In another example, the devices, systems, materials, compounds, compositions, articles, and methods described herein may also be used to perform surgical procedures, such as severing the stapedius tendon using a steerable cannula or wire.

In one aspect, this disclosure is directed to a system for precisely delivering a therapeutic agent to the cochlea of a patient to treat an ear condition. The system can include a body configured to releasably couple with an endoscope, the body defining: (i) a first lumen configured to slidably receive a shaft of the endoscope and (ii) a second lumen; a tube affixed to the body and defining a third lumen that is continuous with the second lumen of the body; a cannula configured to be slidably disposed in the third lumen of the tube and defining a fourth lumen; and a cannula handle affixed to a proximal end of the cannula and defining a fifth lumen that is continuous with the fourth lumen of the cannula. While the cannula is disposed within the third lumen of the tube, the cannula and the cannula handle are slidable, distally and proximally, relative to the body between: (i) a distal travel limit at which a first portion of the cannula handle abuts a first portion of the body and (ii) a proximal travel limit at which a second portion of the cannula handle abuts a second portion of the body.

Such a system for precisely delivering a therapeutic agent to the cochlea may optionally include one or more of the following features. The system may also include the endoscope. The third lumen of the tube may extend along the first lumen of the body. The third lumen of the tube may extend parallel to the first lumen of the body. A distal tip portion of the cannula may include a curved portion. The curved portion of the cannula may be curved between 70° and 110°. The curved portion of the cannula may be compliant and resilient such that: (i) the curved portion straightens to conform to the third lumen of the tube when the curved portion is positioned within the third lumen of the tube and (ii) the curved portion is curved when the curved portion is positioned outside of the third lumen of the tube. A distal tip of the cannula may be fully within the third lumen of the tube while the cannula and the cannula handle are at the proximal travel limit. A distal tip of the cannula may extend distally beyond a distal end of the third lumen of the tube while the cannula and the cannula handle are at the proximal travel limit. The second portion of the body may include a ring, and the cannula handle may be slidably disposed within the ring while the cannula is disposed within the third lumen of the tube.

In another aspect, this disclosure is directed to system for delivering an otic treatment fluid adjacent to a cochlea of a patient. The system includes any embodiment of the system for precisely delivering a therapeutic agent to the cochlea as described herein, and an otic treatment fluid source in fluid communication with the cannula.

In another aspect, this disclosure is directed to a method of treating hearing loss of a patient. The method includes: providing any embodiment of the system for precisely delivering a therapeutic agent to the cochlea as described herein, wherein the endoscope and the cannula are coupled with the body; advancing a distal end portion of the endoscope and a distal end portion of the tube through or around a tympanic membrane of the patient and into a middle ear of the patient; advancing the cannula handle relative to the body to extend a distal tip portion of the cannula from the tube; and delivering, via the cannula, a therapeutic substance into a round window niche of the patient.

Such a method may optionally include one or more of the following features. The therapeutic substance may reside in the round window niche adjacent to a round window membrane of a cochlea of the patient as a gel substance. The delivering the therapeutic substance may be performed while the endoscope provides direct visualization of the round window niche.

In another aspect, this disclosure is directed to a system that includes a middle ear visualization device deliverable through or around a tympanic membrane and having a distal end positionable in a middle ear to visualize a round window niche of a cochlea, and a treatment instrument releasably coupleable to the middle ear visualization device and deliverable through the tympanic membrane. The treatment instrument is configured to deliver a therapeutic substance to treat at least one of: hearing loss, tinnitus, balance disorders, vertigo, Meniere's disease, vestibular neuronitis, vestibular schwannoma, labyrinthitis, otosclerosis, ossicular chain dislocation, cholesteatoma, otitis media, middle ear infections, schwannoma, and tympanic membrane perforations.

In another aspect, this disclosure is directed to a system that includes a body configured to releasably couple with an endoscope, a tube affixed to the body, a cannula configured to be slidably disposed in a lumen of the tube, and a cannula handle affixed to a proximal end of the cannula.

Some or all of the embodiments described herein may provide one or more of the following advantages. First, the systems and methods for treating hearing loss, and all other ear disorders as described herein, can include specialized techniques and instruments that can be used to access the round window niche of the cochlea and other areas in the middle ear and inner ear. In some embodiments, the systems and embodiments can be used to precisely deliver a therapeutic formulation into the round window niche.

Second, in some cases the therapeutic formulation delivered using the systems and methods described herein can be, or can become, a gel. As compared to liquid therapeutic agents that tend to drain from the cochlea, the therapeutic formulation in gel form advantageously remains located adjacent to the round window membrane of the cochlea for an extended period of time during which the active ingredient of the therapeutic formulation can be gradually released and diffuse across the round window membrane into the perilymph (within the cochlea). As compared to liquid therapeutic agents, the therapeutic formulation in gel form in some cases requires precise placement in the round window niche to avoid affecting the mobility of middle ear structures like the ossicles, which could lead to temporary but severe conductive hearing loss. This type of extended release of the active ingredient thereby advantageously reduces the needed frequency of re-administrations of therapeutic agents. In addition, the overall efficacy of the treatment provided by the administration of therapeutic formulations in gel form tends to be greater than the administration of therapeutic formulations in liquid form due to longer middle ear residence and increased diffusion into the inner ear. The systems and methods for treating hearing loss, and all other ear disorders as described herein, can also include specialized techniques and instruments that can be used to access the round window niche of the cochlea and to precisely place a solid implant or sustained delivery system across on or across the round window membrane, or to directly deliver therapeutic treatments into the perilymph across the round window membrane. The systems and methods for treating hearing loss, and all other ear disorders as described herein, can also include specialized techniques and instruments that can be used to precisely deliver therapeutics to other parts of the middle ear cavity.

Third, the systems and methods for treating hearing loss described herein deliver a therapeutic formulation under direct visualization. The use of such direct visualization advantageously allows visual confirmation of the proper placement of the therapeutic formulation (e.g., at the round window niche of the cochlea) with a high level of accuracy. The direct visualization also provides additional benefits such as the ability to ascertain visually whether there are any obstructions of the round window that could inhibit the proper delivery of the therapeutic formulation. For example, in some cases the round window is covered by a pseudomembrane that can be altered or moved to allow improved access to the round window membrane. By using the improved instrumentation described herein, the presence of the pseudomembrane can be visually verified, and thereafter physically altered or moved, so that improved and direct access to the round window membrane can be visually verified. In addition, after the therapeutic formulation has been administered adjacent to the round window membrane of the cochlea, direct visualization can be used to verify that the therapeutic formulation is retained in the desired position and manner.

Fourth, the systems and methods for treating hearing loss and other ear disorders as described herein allow direct access to the middle ear cavity through an incision to the tympanic membrane in a suture-less, low impact manner, or using a tympanic membrane flap approach. In some implementations, such direct access can be safer, less invasive, and achieved with no suturing, sealing or patching of the tympanic membrane.

Fifth, the systems and methods for treating hearing loss and other ear disorders as described herein facilitate treatments in a minimally invasive fashion. Such minimally invasive techniques can tend to reduce recovery times, patient discomfort, and treatment costs. Moreover, the methods described herein can be performed using a local anesthetic rather than requiring general anesthesia. Accordingly, the treatment cost, patient risks, and recovery times are further advantageously reduced.

Sixth, the systems described herein can also be used for diagnostic purposes. Such uses can help in procedure planning, change site of care, and potentially improve patient outcomes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a patient in position for the medical procedure for treating hearing loss and other ear disorders as described herein.

FIG. 2 is a schematic illustration of an otic medical procedure being performed on the patient of FIG. 1, in accordance with some embodiments.

FIG. 3 shows a perspective view of a therapeutic substance residing within a round window niche of the patient of FIG. 1, and that was delivered in accordance with one example procedure depicted by FIG. 2.

FIG. 4 shows a side view of an example system for delivering a therapeutic formulation to treat an ear condition of a patient in accordance with some embodiments.

FIG. 5 shows a longitudinal cross-sectional view of a body of the system of FIG. 4.

FIG. 6 is a perspective view of the body of FIG. 5.

FIG. 7 shows a perspective view of a cannula handle of the system of FIG. 4.

FIG. 8 shows a side view of the system of FIG. 4 engaged with an endoscope and arranged in a first configuration.

FIG. 9 shows the assembly of FIG. 8 arranged in a second configuration.

FIG. 10 shows a tube and cannula of the assembly of FIG. 7 arranged in a first configuration.

FIG. 11 shows the tube and cannula of FIG. 10 arranged in a second configuration.

FIG. 12 shows the tube and cannula of FIG. 10 arranged in a third configuration.

Like reference symbols in the various drawings indicate like elements.

### DETAILED DESCRIPTION OF ILLUSTRATIVE EMBODIMENTS

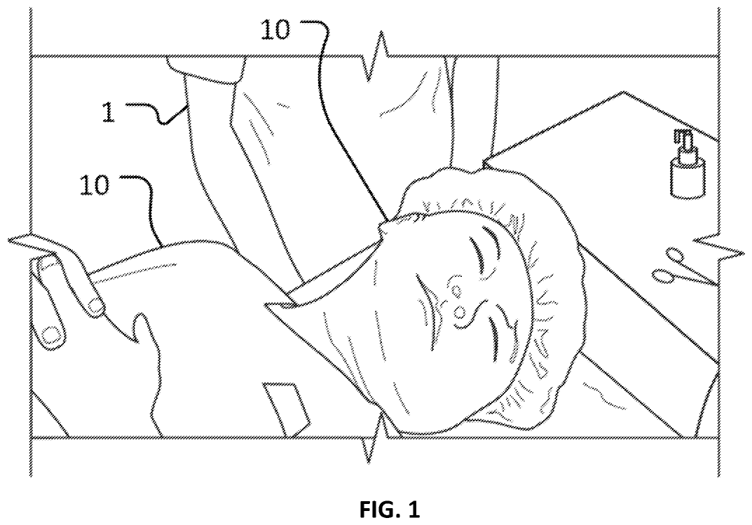

Referring now to FIGS. 1-3, particular example embodiments of systems and methods for treating a patient 10 as described herein can include an improved set of medical instruments for delivering a therapeutic formulation 100 to a targeted site at a cochlea 50 of the patient 10, under direct endoscopic visualization, for example. The devices, systems, and methods described herein can be used to treat and/or prevent a variety of conditions, including but not limited to hearing loss, including hidden hearing loss, noise-induced hearing loss, age-related hearing loss, drug-induced hearing loss (e.g., chemotherapy-induced hearing loss or aminoglycoside-induced hearing loss), sudden sensorineural hearing loss (SSNHL), autoimmune inner ear disease, cholesteatoma, and the like.

While the devices, systems, materials, compounds, compositions, articles, and methods are described herein primarily in the context of treating hearing loss, it should be understood that devices, systems, materials, compounds, compositions, articles, and methods can also be used to treat any other disorder of the middle ear and/or inner ear including, but not limited to, tinnitus, balance disorders including vertigo, Meniere's Disease, vestibular neuronitis, vestibular schwannoma, labyrinthitis, otosclerosis, ossicular chain dislocation, cholesteatoma, middle ear infections, and tympanic membrane perforations, to provide a few examples.

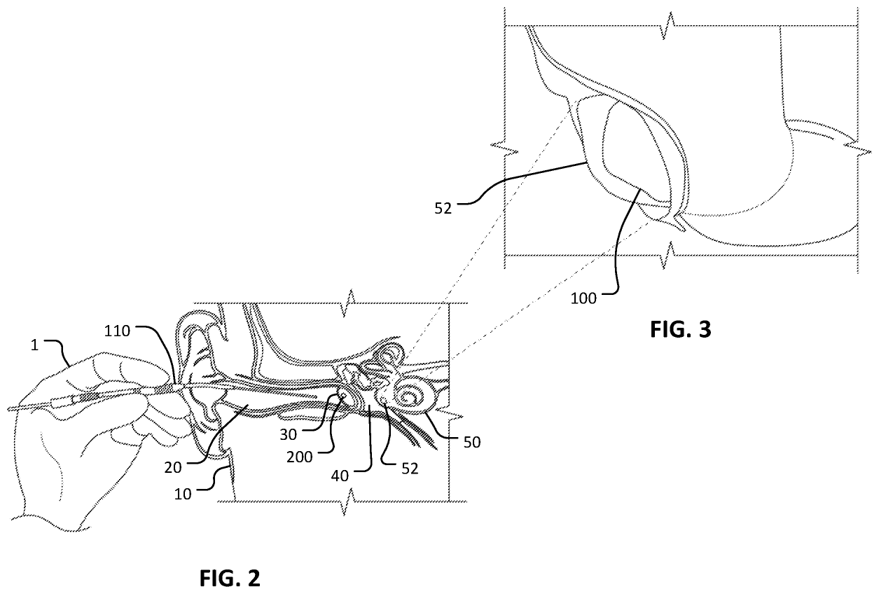

This disclosure describes treatment methods and devices for treating the patient 10 using a minimally invasive approach. As depicted in FIG. 2, a clinician 1 approaches a middle ear 40 via the patient's 10 outer ear canal 20 using one or more instruments (also referred to herein as a “system 110”) as described further below (collectively represented here by a generic instrument 110). In some embodiments, the instruments 110 are advanced through the tympanic membrane (“TM”) 30, via one or more temporarily implanted tympanic membrane port devices 200. In particular embodiments, the instruments 110 are advanced through the TM 30, via one or more incisions in the TM 30. In other embodiments, the instruments 110 are advanced around the TM 30 using a TM flap procedure. Distal end portions of the one or more instruments 110 are thereby advanced into the middle ear 40 of the patient 10. In some embodiments, distal end portions of the one or more instruments 110 are thereby advanced toward a round window niche 52 of the cochlea 50 of the patient 10. The round window niche typically has anterior and posterior pillars or walls on either side, and a bony roof or overhang such that the round window membrane is located within a “cave-like” niche.

As described in more detail below, and as depicted in FIG. 3, in some embodiments the instruments of the system 110 can be configured to achieve a targeted delivery of the therapeutic formulation 100 (or “therapeutic substance 100”) into the round window niche 52 and adjacent to the round window membrane of the cochlea 50. The active ingredient of the therapeutic formulation 100 then moves passively by diffusion across the membrane of the round window 52, according to a concentration gradient, and into the perilymph (within the cochlea 50). In some embodiments, the therapeutic formulation 100 that is delivered adjacent to the round window membrane of the cochlea 50 can thereafter reside adjacent to or within the round window niche 52 as a semi-solid gel substance. As a gel substance, the delivery of the therapeutic formulation 100 will remain in the targeted site at the cochlea 50 so that the therapeutic formulation 100 can gradually release its active ingredient for an extended period of time such as days, weeks, or even months.

After the delivery of the therapeutic formulation 100, the instruments 110 (and the one or more TM port device(s) 200 if used) can be removed from the patient 10. The TM port device(s) 200 can be sized and shaped so that the openings of the TM 30 (in which the TM port device(s) 200 were positioned) can naturally heal (without suturing). The therapeutic formulation 100 (e.g., in gel form) will remain at the targeted site in the round window niche 52 to provide extended therapeutic effects by a controlled, sustained release of the active ingredient into the body of the patient 10.

Sustained release can encompass the release of effective amounts of an active ingredient of the therapeutic formulation 100 for an extended period of time. The sustained release may encompass first order release of the active ingredient, zero order release of the active ingredient, or other kinetics of release such as intermediate to zero order and first order, or combinations thereof. The sustained release may also encompass controlled release of the active ingredient of the therapeutic formulation 100 via passive molecular diffusion driven by a concentration gradient across a membrane or porous structure.

The procedure for delivering the therapeutic formulation 100 into the round window niche 52 of the patient 10 can be repeated periodically as needed for a particular patient's treatment. For example, in some cases deliveries of the therapeutic formulation 100 can be administered about every three to 24 months, each time using the instruments and systems as described herein. In particular cases, an assessment of the patient 10 can be performed to determine whether or when to administer more therapeutic formulation 100. In some cases, a procedure such as magnetic resonance imaging (MRI) (or other type of procedure) can be performed to help make such an assessment.

In FIG. 1, the patient 10 is depicted in an example suitable position and orientation to receive the procedure(s) to treat hearing loss and other ear disorders as described herein. In some cases, the procedure can be performed with the patient 10 fully supine (as shown) or reclined in a chair.

The head of the patient 10 can be rotated to between about 30 to 45 degrees away from the clinician 1 (toward the opposite ear of the patient 10). The jaw of the patient 10 can be slightly elevated, and/or the external portion of the ear of the patient 10 may be pulled superiorly and backward to adjust the canal aperture and angularity. As such, the round window niche 52 of the patient will be oriented generally upward (e.g., away from the ground) so that, upon dispensation of the therapeutic formulation 100 from the delivery instrument, the therapeutic formulation 100 is able to pool at the round window niche 52 and not flow toward the eustachian tube or the ossicular chain.

In some implementations, the patient 10 remains awake during the procedure. That is, the procedure can be performed using a local anesthetic rather than a general anesthetic. For example, in some cases agents such as phenol or lidocaine can be applied to the TM 30 as a local anesthetic to facilitate the procedure. In some cases, the patient 10 can be given general anesthesia for the procedure.

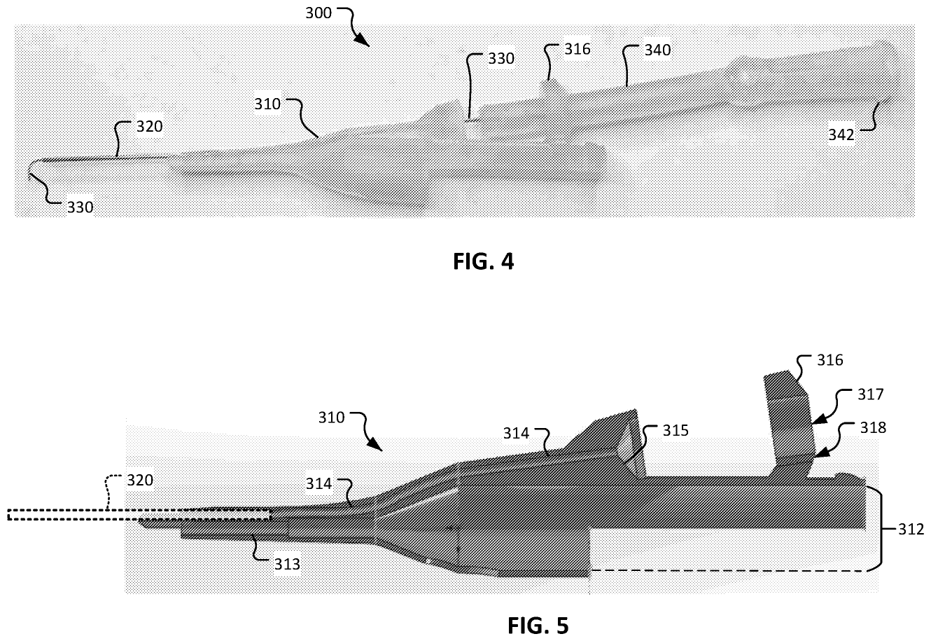

Referring also to FIGS. 4-6, an example system 300 is depicted that can be used to deliver a therapeutic formulation to treat an ear condition of a patient. The system 300 broadly includes a body 310, a tube 320, a cannula 330, and a cannula handle 340. The tube 320 is affixed to, and extends distally from, the body 310. The cannula handle 340 is affixed to a proximal end of the cannula 330. Accordingly, the cannula 330 distally extends from the cannula handle 340.

The body 310 and the tube 320 (which are affixed together) collectively form a first portion of the system 300. The cannula 330 and the cannula handle 340 (which are affixed together) collectively form a second portion of the system 300. The first and second portions of the system 300 can be engaged to each other, and can be disengaged from each other. In FIG. 4, the first and second portions of the system 300 are shown engaged to each other in an operative configuration. In addition, while the first and second portions of the system 300 are engaged to each other, the cannula 330 and cannula handle 340 can be slid (e.g., translated proximally and distally) in relation to the body 310 and the tube 320, as described further below.

In some embodiments, a proximal end of the cannula handle 340 includes a connection configuration 342. The connection configuration 342 can be used to connect a source of an otic treatment fluid (e.g., a therapeutic formulation, a therapeutic substance, etc.) to the cannula handle 340. In some embodiments, the connection configuration 342 is a luer fitting, a luer lock fitting, a threaded fitting, a press-fit connection, and the like. Other styles of fittings can also be used to comprise the connection configuration 342.

FIG. 5 shows a longitudinal cross-sectional view of the body 310. This view allows visualization of various lumens and other features of the body 310. The tube 320 is depicted here in dashed lines.

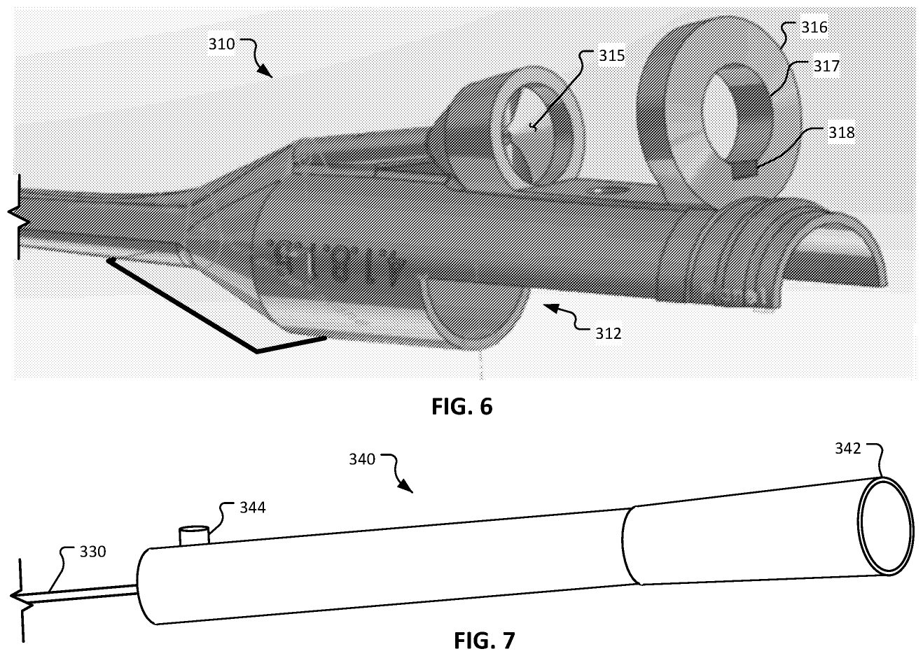

The body 310 defines a space 312 that is configured to receive and releasably couple with an endoscope (e.g., as shown in FIGS. 6-7). The space 312 distally extends to a first lumen 313 that is configured to slidably receive a shaft of the endoscope.

The body 310 also defines a second lumen 314. The second lumen 314 distally extends to, and is continuous with, a lumen defined by the tube 320. Accordingly, the second lumen 314 and the lumen defined by the tube 320 form a single continuous lumen that can slidably receive the cannula 330 (or that can be used for delivery of a therapeutic substance without the cannula 330 disposed therein).

In the depicted embodiment, the first lumen 313 and the lumen defined by the tube 320 are parallel to each other. In some embodiments, the first lumen 313 and the lumen defined by the tube 320 (or at least a distal portion thereof) define a non-zero angle therebetween (e.g., an angle between 0° and 10°, or between 5° and 15°, or between 10° and 20°, or between 0° and 30°, without limitation).

As stated above and described further below, the cannula 330 and cannula handle 340 can be slid (translated proximally and distally) in relation to the body 310 and the tube 320. The system 300 advantageously includes mechanical travel limits to constrain the extent of the proximal and distal movements of the cannula 330 and cannula handle 340 in relation to the body 310 and the tube 320. For example, as shown in FIGS. 5-6, the body 310 includes a distal travel limit 315. When the distal end of the cannula handle 340 is abutted against the distal travel limit 315, the assembly of the cannula 330 and the cannula handle 340 are at their limit of distal travel in relation to the body 310. In addition, the body 310 includes a ring 316 that defines an opening 317 with an adjunct slot 318. The ring 316, in cooperation with the cannula handle 340, provides the proximal travel limit of the cannula 330 and cannula handle 340 in relation to the body 310 and the tube 320. The cannula handle 340 is slidably received inside of the opening 317.

FIG. 7 shows an example cannula handle 340 and the cannula 330 extending distally from the cannula handle 340. The cannula handle 340 defines a lumen that is continuous with the lumen defined by the cannula 330. Accordingly, when a source of a therapeutic substance is coupled to the cannula handle 340 at the connection configuration 342, the therapeutic substance can be pressurized to make it flow into and through the lumen of the cannula handle 340, into and through the lumen of the cannula 330, and out of the distal tip of the cannula 330 to enter a target anatomical location (e.g., a round window niche).

The cannula handle 340 includes a radial projection 344. The projection 344 can be passed through the slot 318 of the body 310 when the cannula handle 340 and the cannula 330 are being initially engaged with the body 310. When the cannula handle 340 and the cannula 330 are engaged with body 310 (in an operative arrangement), the projection 344 resides between the distal travel limit 315 and the ring 316.

The proximal travel limit of the cannula 330 and cannula handle 340 in relation to the body 310 and the tube 320 is provided by the abutment of the projection 344 against the distal-facing surface of the ring 316. That abutment, which limits the proximal travel of the cannula 330 and cannula handle 340, will occur as long as the projection 344 is not aligned with the slot 318.

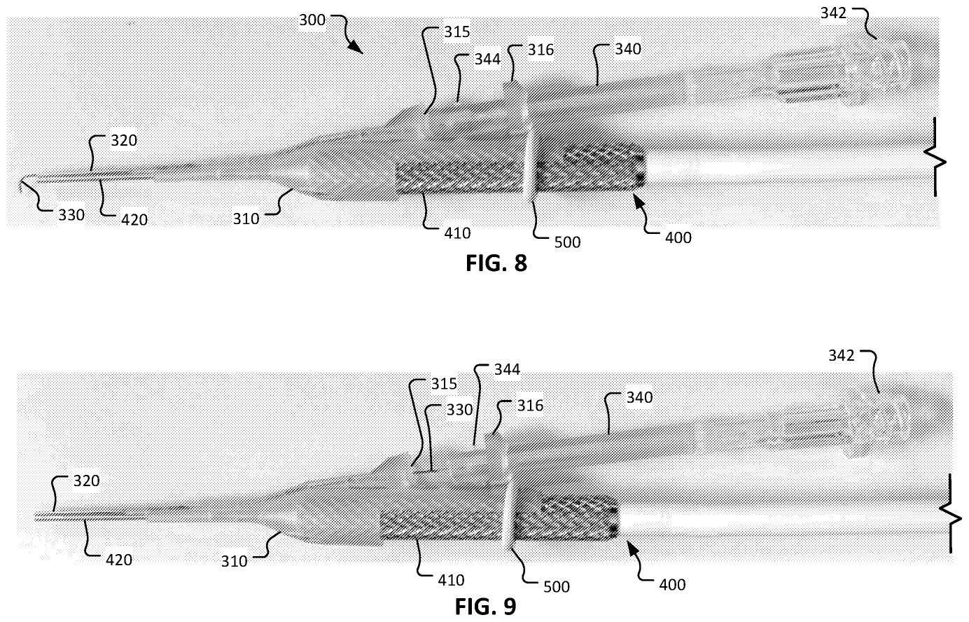

FIGS. 8-9 show an example endoscope 400 releasably coupled to the system 300 in an operative configuration.

The endoscope 400 includes an endoscope handle 410 that is removably engaged within the complementary-shaped space 312 (FIG. 5) defined by the body 310. The endoscope 400 also includes an endoscope shaft 420 extending distally from the endoscope handle 410. The endoscope shaft 420 is removably engaged within the first lumen 313 defined by the body 310, and extends distally from the distal end of the first lumen 313. The endoscope shaft 420 extends along the tube 320 that contains the cannula 330.

The distal tips of the tube 320 and the endoscope shaft 420 are approximately at the same longitudinal location in the depicted embodiment. In some embodiments, the tube 320 extends farther distally than the endoscope shaft 420, for example in a range from 0 mm to 0.5 mm, or from 0.5 mm to 3 mm, or from, 2 mm to 4 mm, or from 3 mm to 15 mm, without limitation. In some embodiments, the endoscope shaft 420 extends farther distally than the tube 320.

Also shown is an elastic compression ring 500. The compression ring 500 provides removable mechanical fixation of the endoscope 400 to the system 300. In particular, the compression ring 500 provides longitudinal and rotational fixation of the endoscope 400 to the system 300. Moreover, in some embodiments the compression ring 500 blocks the slot 318 of the ring 316 (FIG. 6) to prevent inadvertent passage of the projection 344 of the cannula handle 340 through the slot 318. In some embodiments, the compression ring 500 is elastic (e.g., made of silicone in some embodiments) so that the user can selectively rotate the endoscope 400 about its longitudinal axis in relation to the system 300 if so desired.

FIGS. 8-9 show two differing positions of the cannula 330 and cannula handle 340 in relation to the body 310 and the tube 320. In FIG. 8, the cannula 330 and cannula handle 340 are extended farther distally relative to the body 310 and the tube 320 than in FIG. 9. Said another way, in FIG. 9 the cannula 330 and cannula handle 340 have been pulled back in a proximal direction relative to the body 310 and the tube 320 than in FIG. 8. It can be seen that the projection 344 is between the distal travel limit 315 and the proximal travel limit (the distal-facing surface of the ring 316) in both arrangements.

In the arrangement of FIG. 8, a distal portion of the cannula 330 is extending distally of the distal tip of the tube 320. In the arrangement of FIG. 9, that distal portion of the cannula 330 is fully residing within the lumen of the tube 320 and is straightened. In some embodiments, when the cannula 330 and cannula handle 340 are located at their proximal travel limit in relation to the body 310 and the tube 320, a short portion of the distal portion of the cannula 330 extends distally of the distal tip of the tube 320.

It can be seen in FIG. 8 that the distal portion of the cannula 330 is curved or includes a curved portion in this embodiment. In some embodiments, that curved portion of the cannula 330 is created as a result of a heat-set process that induces the curved shape of the distal portion of the cannula 330. That is, the distal portion of the cannula 330 can be constrained in a curved configuration and then heated in order to cause the distal portion of the cannula 330 to remain in a curved configuration after the heat treatment. Moreover, the cannula 330 is compliant and resilient (e.g., super elastic in some embodiments) such that the curved portion readily straightens to conform to the lumen of the tube 320 when the curved portion is positioned within the lumen of the tube 320. The curved portion of the distal portion of the cannula 330 seeks its natural curved shape when the curved portion is positioned outside of the lumen of the tube 320. In some embodiments, the cannula 330 is made of a metallic material such as Nitinol or stainless steel. In some embodiments, the cannula 330 is made of a polymeric material.

While the depicted cannula 330 includes a single curved portion, any number of curves and/or configuration of curves can be engendered to the cannula 330. Such a curved portion can be advantageous for approaching particular anatomical features of a patient. In one such example, the curved portion can be advantageous for orienting the distal end portion of the cannula 330 to approach and/or to enter into the round window niche. Thereafter, a therapeutic substance can be injected into the round window niche via the cannula 330.

In another example, in some embodiments the cannula 330 can include multiple curves (e.g., an S-shape, and the like). Such a cannula 330 with multiple curves can be advantageous for entering and injecting a therapeutic substance into a scala tympani of a cochlea, for example.

In some embodiments, the cannula 330 can include one or more compound curves, curves that are in different planes, curves that have differing radii, one or more curves at any desired angle, and any other desired features or configurations.

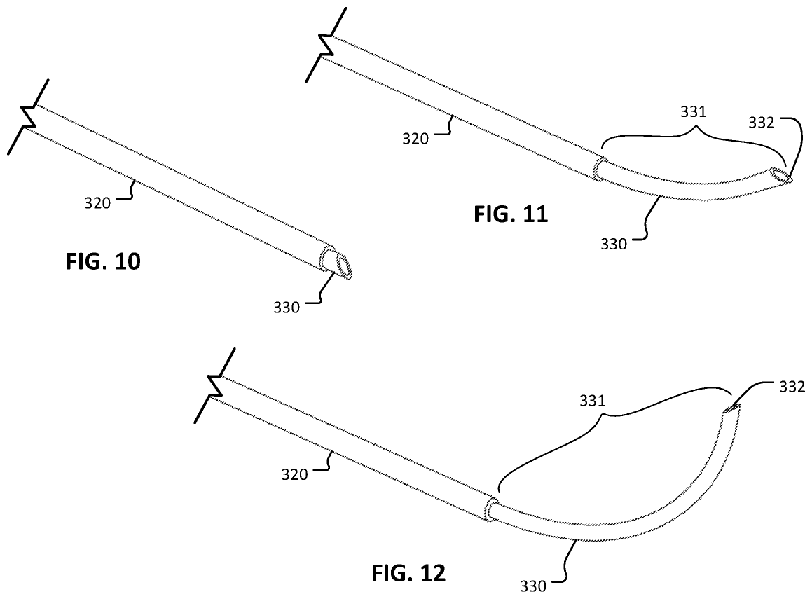

FIGS. 10-12 show the tube 320 and the cannula 330 in various arrangements. In FIG. 10, the cannula 330 is only slightly extended from the distal tip of the tube 320. In FIG. 11, the cannula 330 is extended from the distal tip of the tube 320 by a first distance. In FIG. 12, the cannula 330 is extending from the distal tip of the tube 320 by a second distance that is greater than the first distance.

In this example, a distal end portion of the cannula 330 is curved. The curve manifests as the distal end portion of the cannula 330 emerges from the distal tip of the tube 320. For example, in FIG. 11 the curved distal end portion of the cannula 330 is partially within the tube 320 and partially outside of the tube 320. Accordingly, the distal end portion 331 of the cannula 330 that is exposed from the distal tip of the tube 320 exhibits a fraction of the total curve that the distal end portion of the cannula 330 is capable of exhibiting. In contrast, in FIG. 12 the distal end portion 331 of the cannula 330 that is exposed from the distal tip of the tube 320 includes the entire curved distal end portion of the cannula 330.

The various configurations of the tube 320 and the cannula 330 shown in FIGS. 10-12 illustrate how the angulation of the distal end portion 331 of the cannula 330 can be controlled as desired. That is, the angle of the distal end portion 331 of the cannula 330 can be adjusted by controlling the distance that the distal end portion 331 of the cannula 330 is exposed from the distal tip of the tube 320. Moreover, the user can also rotate the cannula 330 about its longitudinal axis in relation to the tube 320. Accordingly, the user can effectively steer the cannula 330 by controllably varying such physical aspects of the cannula 330 relative to the tube 320.

In some embodiments, the curved distal end portion of the cannula 330 extends along an angle that is about 90°. In some embodiments, the curved distal end portion of the cannula 330 extends along an angle that is between 70° and 110°, or between 60° and 120°, or between 0 to 60°. In some embodiments, the cannula 330 is straight and has no curve. In particular embodiments, the curved distal end portion of the cannula 330 is only slightly curved, e.g., extends along an angle that is between 0° and 10°, or between 5° and 20°, without limitation. The curved distal end portion of the cannula 330 can have a bend with a radius in a range of about 1.75 mm to 2.0 mm in some embodiments. In some embodiments, the bend in the curved distal end portion of the cannula 330 has a radius that is between about 1 mm and 3 mm, or between 1 mm and 5 mm, or between 1.5 mm to 6.0 mm, without limitation. In some embodiments, the very distal end of the cannula 330 can be linear, extending straight from the bend to the tip by approximately 1 mm, or 0 mm to 1 mm, or 1 mm to 3 mm, or 2 mm to 5 mm, without limitation. Specific combinations of bend radius and distal tip extension can be beneficial for accessing specific anatomic features, such as the round window niche, in order to have the distal tip of the cannula 330 extend into targeted locations in said features without being blocked or interfered with by surrounding structures (such as the anterior and posterior pillars in the case of the round window niche).

In some embodiments, the cannula 330 has an outer diameter of about 0.2 mm. In particular embodiment, the cannula 330 has an outer diameter that is between 0.1 mm and 0.3 mm, or between 0.1 mm and 0.5 mm, without limitation. The tube 320 can have any desired size. In some embodiments, the tube 320 is a 24 gauge stainless steel hypotube. In particular embodiments, the tube 320 is a stainless steel or nitinol hypotube between 20 and 28 gauge, without limitation.

The cannula 330 has a distal tip 332. In some cases, the distal tip 332 can be used to manipulate tissue during a medical procedure. Accordingly, the distal tip 332 can be configured in various ways to customize the cannula 330 for its intended use. In some embodiments, such as the depicted embodiment, the distal tip 332 can include a point that can puncture tissue (e.g., tissue such as a pseudo membrane of a round window niche). In certain embodiments, the distal tip 332 can include a hook, a slot, teeth, a notch, a biased or angled cut as shown (of varying angles relative to the cannula bend), and the like to make the distal tip 332 suitable for puncturing and/or retracting tissue. In particular embodiments, the distal tip 332 can be an atraumatic tip to prevent inadvertent infliction of injury to tissues.

In some embodiments, the distal travel limiter 315 and the ring 316 are joined to form a continuous cylinder, or partial cylinder. This cylinder can include an open slot running longitudinally down the cylinder. The projection 344 can then be configured to advance or translate proximally and distally within this slot, with the length of the slot minus the longitudinal length of the projection dictating the length of allowable travel of the cannula 330 and cannula handle 340. At the same time, the width of the slot defined by the cylinder wall can allow for controlled range of rotation of the cannula 330 about its longitudinal axis. The cylinder slot and travel limiter (whether it be a pin as shown, or longer “fin” running along the axis of the cannula handle 340) can have dimensions that are configured to allow only the necessary movement of the cannula 330 along the longitudinal axis and rotationally with respect to the endoscope 400. The rotation can be set, for example, to limit the rotational range of motion to within a total range of 20° to 60°, or 40° to 80°, or 60° to 100°, or 80° to 120°, or 100° to 140°, or 120° to 160°, or 140° to 180°, without limitation, such that it allows for a range of movement within the visual field of the endoscope 400. This minimizes unintended movement of the cannula 330 outside of endoscope view which may result in local tissue damage. This also prevents the orientation of the cannula 330 from inadvertently becoming misplaced during transit and assembly.

### Additional Features and or Embodiments

While the embodiments described above have the tube 320 affixed to the body 310, in some embodiments the tube 320 is slidable relative to the body 310. An injection of therapeutic agent can be made via such a tube 320, or the tube 320 can contain the cannula 330 as described above. In such an embodiment the slidable advancement of tube 320 can be via an additional handle, through a slider in handle 310, or other configurations.

In some configurations, the tip of tube 320 can itself have features to manipulate tissue during a medical procedure. In such a configuration the distal tip can include a point, a hook, a slot, teeth, a notch, a biased or angled cut, and the like.

In some embodiments, the tube 320 can include a surface treatment (e.g. roughening, diamond dusting) to increase engagement with membranous tissue and aid in manipulation and/or dissection.

In some embodiments, a tube 320 is absent, and the handle 310 itself comprises the totality of the channel 314 that can be either itself used to deliver fluid agent, or act as a throughway for a cannula 330 or other instruments. While the embodiments described above have the member 330 described as a cannula 330, in some embodiments a wire can replace the cannula 330. In some such embodiments the wire can be attached to the handle 340, or the wire can pass through a luminal space defined inside a handle 340 and optionally be attached to its own handle. The wire can be loaded into a handle at the time of use and transiently bonded to a handle 340, or can be supplied loaded and transiently or permanently bonded to handle 340. The wire can slidably interact with the handle 310 and tube 320, and its sliding and/or rotation be controlled by its handle 340. In some embodiments, the wire can have a curve in its distal end portion (e.g., similar to as shown in FIG. 12). As an example use, the wire can be used to interact with the tissue, and fluid delivery can be delivered via the luminal space 314 contiguous with tube 320 after the wire is removed. The wire can be partially retracted to aid fluid delivery or totally removed. Alternatively the fluid delivery can be via the annular space 314 around the wire. The handle 310 can incorporate a Y connector to allow simultaneous attachment of a fluid delivery method to throughway 314 and wire management.

Although the embodiments have been predominantly described in a modular form that can attach or affix to an endoscope, it can be envisioned by the inventors that elements of, or the entirety of, embodiments described can be incorporated into the endoscope to varying degrees (such as using a slidable cannula with nitinol wire in combination with an endoscope with an integrated working channel).

While the devices, systems, materials, compounds, compositions, articles, and methods described herein described in the context of treating hearing loss, it should be understood that the devices, systems, materials, compounds, compositions, articles, and methods may be used to treat any disorder of the middle ear and/or inner ear including, but not limited to, tinnitus, balance disorders including vertigo, Meniere's Disease, vestibular neuronitis, vestibular schwannoma, labyrinthitis, otosclerosis, ossicular chain dislocation, cholesteatoma, otitis media, middle ear infections, and tympanic membrane perforations, to provide a few examples.

Although the round window membrane is one target site for therapeutic agent delivery or access, the systems and methods described herein can also be used for precise delivery of therapeutic agents to other target sites, such as the oval window or other parts of the middle ear cavity, and for providing access to other features or regions of the middle ear. For example, the systems and methods described herein can be used for minimally invasive surgical reconstruction of the ossicular chain, for removal of cholesteatoma, for diagnostic assessment, and other procedures. Any and all such techniques for using the systems and methods described herein are included within the scope of this disclosure.

The devices, systems, materials, compounds, compositions, articles, and methods described herein may be understood by reference to the above detailed description of specific aspects of the disclosed subject matter. It is to be understood, however, that the aspects described above are not limited to specific devices, systems, methods, or specific agents, as such may vary. It is also to be understood that the terminology used herein is for the purpose of describing particular aspects only and is not intended to be limiting.

A number of embodiments have been described. Nevertheless, it will be understood that various modifications may be made without departing from the claim scope here. Additionally, aspects of the various embodiments can be combined with other aspects of other embodiments, without limitation. Accordingly, other embodiments, including hybrid embodiments, are within the scope of this disclosure.

1. A system for delivering a therapeutic formulation to treat an ear condition of a patient, the system comprising:
a body configured to releasably couple with an endoscope, the body defining: (i) a first lumen configured to slidably receive a shaft of the endoscope and (ii) a second lumen;
a tube affixed to the body and defining a third lumen that is continuous with the second lumen of the body;
a cannula configured to be slidably disposed in the third lumen of the tube and defining a fourth lumen; and
a cannula handle affixed to a proximal end of the cannula and defining a fifth lumen that is continuous with the fourth lumen of the cannula,
wherein, while the cannula is disposed within the third lumen of the tube, the cannula and the cannula handle are slidable, distally and proximally, relative to the body between: (i) a distal travel limit at which a first portion of the cannula handle abuts a first portion of the body and (ii) a proximal travel limit at which a second portion of the cannula handle abuts a second portion of the body.

2. The system of claim 1, further comprising the endoscope.

3. The system of claim 1, wherein the third lumen of the tube extends along the first lumen of the body.

4. The system of claim 3, wherein the third lumen of the tube extends parallel to the first lumen of the body.

5. The system of claim 1, wherein a distal tip portion of the cannula comprises a curved portion.

6. The system of claim 5, wherein the curved portion of the cannula is curved between 70° and 110°.

7. The system of claim 5, wherein the curved portion of the cannula is compliant and resilient such that: (i) the curved portion straightens to conform to the third lumen of the tube when the curved portion is positioned within the third lumen of the tube and (ii) the curved portion is curved when the curved portion is positioned outside of the third lumen of the tube.

8. The system of claim 4, wherein a distal tip of the cannula is fully within the third lumen of the tube while the cannula and the cannula handle are at the proximal travel limit.

9. The system of claim 4, wherein a distal tip of the cannula extends distally beyond a distal end of the third lumen of the tube while the cannula and the cannula handle are at the proximal travel limit.

10. The system of claim 1, wherein the second portion of the body comprises a ring, and wherein the cannula handle is slidably disposed within the ring while the cannula is disposed within the third lumen of the tube.

11. The system of claim 1, further comprising:
an otic treatment fluid source in fluid communication with the cannula.

12. A method of using the system of claim 1 for treating hearing loss of a patient, the method comprising:
while an endoscope and the cannula are coupled with the body, advancing a distal end portion of the endoscope and a distal end portion of the tube through or around a tympanic membrane of the patient and into a middle ear of the patient;
advancing the cannula handle relative to the body to extend a distal tip portion of the cannula from the tube; and
delivering, via the cannula, a therapeutic substance into a round window niche of the patient.

13. The method of claim 12, wherein the therapeutic substance resides in the round window niche adjacent to a round window membrane of a cochlea of the patient as a gel substance.

14. The method of claim 12, wherein the delivering the therapeutic substance is performed while the endoscope provides direct visualization of the round window niche.

15. A system comprising:
a middle ear visualization device deliverable through or around a tympanic membrane and having a distal end positionable in a middle ear to visualize a round window niche of a cochlea; and
a treatment injection shaft coupled side-by-side with the middle ear visualization device and deliverable through the tympanic membrane such that a distal tip of the treatment injection shaft is positioned distally of the distal end of the middle ear visualization device, the treatment injection shaft configured to deliver a therapeutic substance to treat at least one of: hearing loss, tinnitus, balance disorders, vertigo, Meniere's disease, vestibular neuronitis, vestibular schwannoma, labyrinthitis, otosclerosis, ossicular chain dislocation, cholesteatoma, otitis media, middle ear infections, schwannoma, and tympanic membrane perforations.

16. The system of claim 15, further comprising:
a body that defines: (i) a first lumen configured to slidably receive a shaft of an endoscope of the middle ear visualization device and (ii) a second lumen;
a tube affixed to the body wherein;
the treatment injection shaft is slidably disposed in the tube; and
a proximal handle at a proximal end of the treatment injection shaft so that the proximal handle engages with the body both at a distal travel limit and a proximal travel limit.

17. The system of claim 16, wherein, the of the treatment injection shaft is slidable within the tube, distally and proximally, relative to the body between: (i) the distal travel limit at which a first portion of the proximal handle abuts a first portion of the body and (ii) the proximal travel limit at which a second portion of the proximal handle abuts a second portion of the body.

18. The system of claim 16, further comprising the endoscope.

19. A system comprising:
a body configured to releasably couple with an endoscope;
a tube affixed to the body;
a cannula configured to be slidably disposed in a lumen of the tube; and
a cannula handle affixed to a proximal end of the cannula.

20. The system of claim 19, further comprising a source of an otic treatment fluid in communication with the cannula so as to deliver from a cannula an otic treatment fluid to treat at least one of: hearing loss, tinnitus, balance disorders, vertigo, Meniere's disease, vestibular neuronitis, vestibular schwannoma, labyrinthitis, otosclerosis, ossicular chain dislocation, cholesteatoma, otitis media, middle ear infections, schwannoma, and tympanic membrane perforations.
